(19) United States
(12) Patent Application Publication
(10) Pub. No.: US 2010/0222623 A1
(43) Pub. Date: Sep. 2, 2010

(54) Method for selectively oxidizing ethane to ethylene

(76) Inventor: Debra A Ryan, League City, TX (US)

Correspondence Address: Celanese International Corporation;MARTIN CONNAUGHTON, 9502 BAYPORT BLVD., CLEAR LAKE TECHNICAL CENTER, PASADENA, TX, 77507, US

(21) Appl. No.: 11/920,815

(22) PCT Filed: Apr. 28, 2006
(86) PCT No.: PCT/US2006/016458

§ 371 Date: Nov. 20, 2007

Related U.S. Application Data
(60) Provisional application No. 60/686,099, filed on Jun. 1, 2005.

(51) Int. Cl. C07C 5/333

(52) U.S. Cl. 585/654

(57) ABSTRACT

A process is disclosed for selectively preparing ethylene by oxidizing ethane in the presence of oxygen using a catalyst having the formula MoₐVᵥTaₓTe_{y}. Preferably a is 1.0; v is about 0.01 to about 1.0; x is about 0.01 to about 1.0; and y is about 0.01 to about 1.0.

### FIELD OF THE INVENTION

The invention relates to the production of ethylene. In particular, a method of selectively oxidizing ethane to ethylene using a mixed oxide catalyst containing vanadium and tungsten or molybdenum is disclosed.

### BACKGROUND OF THE INVENTION

The oxidative dehydrogenation of ethane to ethylene in the gas phase at temperatures above 500° C. has been discussed, for example, in U.S. Pat. Nos. 4,250,346, 4,524,236, and 4,568,790.

U.S. Pat. No. 4,250,346 describes the use of a catalyst composition containing the elements molybdenum, X and Yin the ratio a:b:c for oxidation of ethane to ethylene, where X is Cr, Mn, Nb, Ta, Ti, V and/or W, and Y is Bi, Ce, Co, Cu, Fe, K, Mg, Ni, P, Pb, Sb, Si, Sn, Ti and/or U, and a is 1, b is from 0.05 to 1, and c is from 0 to 2. The total value of c for Co, Ni and/or Fe must be less than 0.5. The reaction is carried out in the gas phase at temperature below about 550° C. The efficiency of the conversion to ethylene ranges from 50 to 94%, depending upon ethane conversion. The catalysts disclosed can likewise be used for the oxidation of ethane to acetic acid, the efficiency of the conversion to acetic acid being about 18%, with an ethane conversion of 7.5%. Reaction pressures are very low, generally 1 atm, which restricts productivity and commercial viability.

U.S. Pat. No. 4,568,790 describes a process for oxidizing ethane to ethylene using an oxide catalyst containing Mo, V, Nb, and Sb. The reaction is preferably carried out at about 200° C. to about 450° C. The calculated selectivity for ethylene at 50% conversion of ethane ranges from 63 to 76%. Again low reaction pressures limit usefulness.

U.S. Pat. No. 4,524,236 describes a process for oxidizing ethane to ethylene using an oxide catalyst containing Mo, V, Nb, and Sb and at least one metal from the group consisting of Li, Sc, Na, Be, Mg, Ca, Sr, Ba, Ti, Zr, Hf, Y, Ta, Cr, Fe, Co, Ni, Ce, La, Zn, Cd, Hg, Al, Tl, Pb, As, Bi, Te, U, and W. The reaction is preferably carried out at 200° C. to about 400° C. The selectivity for ethylene at 51% conversion of ethane is as high as 80% for one of the compositions discussed in the '236 patent, but productivity is low.

The above-mentioned specifications are principally concerned with the preparation of ethylene. The use of mixed metal oxide catalysts to convert ethane to acetic acid is also known. For example, U.S. Pat. No. 5,162,578 describes a process for the selective preparation of acetic acid from ethane, ethylene or mixtures thereof with oxygen in the presence of a catalyst mixture which comprises at least: (A) a calcined catalyst of the formula MoₓV_{y} or MoₓV_{y}Z_{y}, in which Z can be one or more of the metals Li, Na, Be, Mg, Ca, Sr, Ba, Zn, Cd, Hg, Sc, Y, La, Ce, Al, Tl, Ti, Zr, Hf, Pb, Nb, Ta, As, Sb, Bi, Cr, W, U, Te, Fe, Co and Ni, and x is from 0.5 to 0.9, y is from 0.1 to 0.4, and z is from 0.001 to 1, and (B) an ethylene hydration catalyst and/or ethylene oxidation catalyst. The second catalyst component B is, in particular, a molecular sieve catalyst or a palladium-containing oxidation catalyst. The catalyst mixture was used to produce acetic acid and ethylene from a feed gas mixture consisting of ethane, oxygen, nitrogen and steam. The acetic selectivity was 34% and the ethylene selectivity was 62% with an ethane conversion of 4%. The high conversion rates of ethane were only achieved with the catalyst mixture described, but not in a single catalyst comprising components A and B.

A further process for the preparation of a product comprising ethylene and/or acetic acid is described in European Patent No. EP 0 407 091 B1. According to this process, ethane and/or ethylene and a gas containing molecular oxygen is brought into contact at elevated temperature with a mixed metal oxide catalyst composition of the general formula AₐX_{b}Y_{c} in which A is Mo_{d}ReₑW_{f}; X is Cr, Mn, Nb, Ta, Ti, V and/or W; Y is Bi, Ce, Co, Cu, Fe, K, Mg, Ni, P, Pb, Sb, Si, Sn, Tl and/or U; a is 1; b and c are independently 0 to 2; d+e+f=a, and e is nonzero. The selectivity for acetic acid or ethylene could be adjusted by adjusting the ratio of Mo to Re. The maximum selectivity obtained for acetic acid was 78% at 14.3% ethane conversion. The highest selectivity for ethylene was 70% at 15% ethane conversion.

It is therefore an object of the invention to provide a process that allows ethane and/or ethylene to be oxidized to ethylene in a simple and targeted manner and at high selectivity and space-time yield under reaction conditions which are as mild as possible.

### SUMMARY OF THE INVENTION

It has surprisingly been found that it is possible to oxidize ethane to ethylene under relatively mild conditions in a simple manner at high selectivity and excellent space-time yields when using a catalyst having the formula MoₐVᵥTaₓTe_{y}. Preferably a is 1.0; v is about 0.01 to about 1.0, more preferably about 0.1 to about 0.5; x is about 0.01 to about 1.0, more preferably about 0.05 to about 0.2; and y is about 0.01 to about 1.0, more preferably about 0.1 to about 0.5.

A further aspect of the invention provides a catalyst particularly suited for oxidizing ethane to produce ethylene. According to the particularly preferred embodiment, the catalyst has the formula Mo_{1.0}V_{0.3}Ta_{0.1}Te_{0.3}O_{z}, where z depends on the oxidation state of the metals and is the number that renders the catalyst electronically neutral.

### DETAILED DESCRIPTION OF THE INVENTION

The present invention provides a process for selectively preparing ethylene from a gaseous feed comprising ethane and oxygen, by bringing the gaseous feed into contact with catalyst having the formula MoₐVᵥTaₓTe_{y}. Preferably a is 1.0; v is about 0.01 to about 1.0, more preferably about 0.1 to about 0.5; x is about 0.01 to about 1.0, more preferably about 0.05 to about 0.2; and y is about 0.01 to about 1.0, more preferably about 0.1 to about 0.5. As used herein, the catalyst is referred to using the formula MoₐVᵥTaₓTe_{y}. One of skill in the art will appreciate that the catalyst is actually a mixed oxide having the formula MoₐVᵥTaₓTe_{y} O_{z}. The amount of oxygen, z, is determined by the oxidation states of A, V, Ta, and Te and cannot be generally specified.

According to a preferred embodiment, the catalyst has the formula MoₐVᵥTaₓTe_{y}O_{z} wherein a, v, x, and y have the ranges specified above. A particularly preferred catalyst has the formula Mo_{1.0}V_{0.3}Ta_{0.1}Te_{0.3}O_{z}.

The catalyst of the invention can be prepared as described in U.S. Pat. No. 6,653,253, by Lin, the entire contents of which are incorporated herein by reference. Briefly, metal compounds that are the sources of the metals in the catalyst are combined in at least one solvent in appropriate amounts to form a solution. Generally, the metal compounds contain elements A, V, X, Y, and at least one of the metal compounds contains O. For example, a compound according to AₐVᵥXₓY_{y}O wherein A is Mo, X is Ta, and Y is Te, can be prepared by combining an aqueous solution of tantalum oxalate with an aqueous solution or slurry of ammonium heptamolybdate, ammonium metavanadate and telluric acid, wherein the concentrations of the metal compounds are such that the atomic ratio of the respective metal elements are in the proportions prescribed by the stoichiometry of the target catalyst.

Additionally, a wide range of materials including, oxides, nitrates, halides or oxyhalides, alkoxides, acetylacetonates, and organometallic compounds may be used. For example, ammonium heptamolybdate may be used as the source of molybdenum in the catalyst. However, compounds such as MoO₃, MoO₂, MoCl₅, MoOCl₄, Mo(OC₂H₅)₅, molybdenum acetylacetonate, phosphomolybdic acid and silicomolybdic acid may also be utilized instead of ammonium heptamolybdate. Similarly, ammonium metavanadate may be used as the source of vanadium in the catalyst. However, compounds such as V₂O₅, V₂O₃, VOCl₃, VCl₄, VO(OC₂H₅), vanadium acetylacetonate and vanadyl acetylacetonate may also be utilized instead of ammonium metavanadate. The tellurium source may include telluric acid, TeCl₄, Te(OC₂H₅)₅, Te(OCH(CH₃)₂)₄ and TeO₂. The tantalum source may include ammonium tantalum oxalate, Ta₂O₅, TaCl₅, tantalic acid or Ta(OC₂H₅)₅ as well as the more conventional tantalum oxalate.

Suitable solvents include water, alcohols (including but not limited to methanol, ethanol, propanol, and diols etc.) as well as other polar solvents known in the art. Generally, water is preferred. The water is any water suitable for use in chemical synthesis including, without limitation, distilled water and deionized water. The amount of water present is that amount sufficient to keep the elements substantially in solution long enough to avoid or minimize compositional and/or phase segregation during the preparation steps. Once the aqueous solution is formed, the water is removed by a combination of any suitable methods known in the art to form a catalyst precursor. Such methods include, without limitation, vacuum drying, freeze drying, spray drying, rotary evaporation, and air drying. Rotary evaporation or air drying are generally preferred.

Once obtained, the catalyst precursor is calcined under an inert atmosphere. The inert atmosphere may be any material which is substantially inert to, i.e., does not react or interact with, the catalyst precursor. Suitable examples include, without limitation, nitrogen, argon, xenon, helium or mixtures thereof. Preferably, the inert atmosphere is argon or nitrogen, more preferably argon. The inert atmosphere may or may not flow over the surface of the catalyst precursor. Typically, if nitrogen is used, flowing is used. If the inert atmosphere is argon, then typically flowing is not used. When the inert atmosphere does flow over the surface of the catalyst precursor, the flow rate can vary over a wide range, for example, at a space velocity from 1 to 500 hr⁻¹. The calcination is typically done at a temperature of from 350° C. to 850° C., preferably from 400° C. to 700° C., more preferably from 500° C. to 640° C. The calcination is performed for long enough to form the catalyst. In one embodiment, the calcination is performed from 0.5 to 30 hours, preferably from 1 to 25 hours and more preferably from 1 to 15 hours.

The catalyst of the invention may be used as a solid catalyst alone or may be used with a suitable support. Conventional support materials are suitable, for example, porous silicon dioxide, ignited silicon dioxide, kieselguhr, silica gel, porous or nonporous aluminum oxide, titanium dioxide, zirconium dioxide, thorium dioxide, lanthanum oxide, magnesium oxide, calcium oxide, barium oxide, tin oxide, cerium dioxide, zinc oxide, boron oxide, boron nitride, boron carbide, boron phosphate, zirconium phosphate, aluminum silicate, silicon nitride or silicon carbide, but also glass, carbon-fiber, carbon, activated carbon, metal-oxide or metal networks or corresponding monoliths.

Support materials should be chosen based on optimizing both the surface area and pore size for the specific oxidation of interest. The catalyst can be employed after shaping as a regularly or irregularly shaped support element, but also in powder form as a heterogeneous oxidation catalyst.

Alternatively, the catalyst of the invention may be encapsulated in a material. Suitable materials for encapsulation include SiO₂, P₂O₅, MgO, Cr₂O₃, TiO₂, ZrO₂, and Al₂O₃. Methods of encapsulating materials in oxides are known in the art. A suitable method of encapsulating materials in oxides is described in U.S. Pat. No. 4,677,084 and references cited therein, the entire contents of which are incorporated herein by references.

The oxidation of ethane can be carried out in a fluidized bed or in a fixed bed reactor. For use in a fluidized bed, the catalyst is normally ground to a particle size in the range from 10 to 200 μm or prepared by spray drying.

The gaseous feedstock, and any recycle gas combined with said feedstock gas, contains primarily ethane but may contain some amount of ethylene, and is fed to the reactor as a pure gas or in a mixture with one or more other gases. Suitable examples of such additional or carrier gases are nitrogen, methane, carbon monoxide, carbon dioxide, air and/or steam. The gas containing molecular oxygen may be air or a gas which has a higher or lower molecular oxygen concentration than air, for example pure oxygen.

The reaction is generally carried out at about 200 to about 500° C., preferably about 200 to about 400° C. The pressure can be atmospheric or superatmospheric, for example about 1 to about 50 bar, preferably about 1 to about 30 bar.

The reaction can be carried out in a fixed bed or fluidized bed reactor. Ethane can be first mixed with an inert gas such as nitrogen or steam before oxygen or the gas containing molecular oxygen is fed in. The mixed gases can be preheated to the reaction temperature in a preheating zone before the gas mixture is brought into contact with the catalyst. Acetic acid can be removed from the gas leaving the reactor by condensation. The other gases can be returned to the reactor inlet, where oxygen or the gas containing molecular oxygen, and ethane is metered in.

According to a preferred embodiment, ethane feed is purified and distilled to provide purified ethane as a top stream and propane and other heavies as a bottom stream. The ethane is provided to an oxidation reactor, which is a fluidized bed reactor utilizing the catalyst described above. According to a particularly preferred embodiment, the catalyst has the formula MoₐVᵥTaₓTe_{y}O_{z}, where a, v, x, y, and z are as defined above. According to an especially preferred embodiment, the catalyst has the formula Mo_{1.0}V_{0.3}Ta_{0.1}Te_{0.3}O_{z}. Oxygen is also provided to the reactor.

The oxidation reaction produces a mixture of gases including ethylene, acetic acid, water, COₓ (CO and CO₂), unreacted ethane, and assorted heavy by-products. The product gas effluent from the reactor is preferably filtered to remove catalyst fines and is then routed to a recycle gas scrubber, which produces a top stream containing ethylene, ethane, and COₓ. The top stream from the recycle gas scrubber is routed to a fixed bed CO converter followed by a processing step that removes the COₓ from the top stream. The stream is then routed to an ethylene purification tower that provides product ethylene as a top stream and ethane as a bottom stream, which is recycled to the oxidation reactor.

The bottom stream from the recycle gas scrubber, which contains acetic acid, water, and heavy ends by-products, may be purified as known in the art to provide purified acetic acid. For example, the bottom stream may be routed to a drying column to remove water followed by a heavy ends column to remove propionic acid and other heavy components.

One of skill in the art will appreciate that the towers, scrubbers, and routing referred to in the preceding paragraphs will have associated with them various heat exchangers, pumps, and connectors and will have operating parameters that are determined by the particular mixture of gases involved. It is within the ability of one of ordinary skill in the art to determine the proper configurations and parameters, given the above disclosure.

A further aspect of the invention is a catalyst that is particularly suitable for the oxidation of ethane to produce ethylene and acetic acid with a high selectivity for ethylene. Preferably, the selectivity for ethylene is about 80%, more preferably about 70% to about 80%. According to a preferred embodiment, the catalyst has the formula MoₐVᵥTaₓTe_{y}O_{z}, where a, v, x, y, and z are as defined above. According to particularly preferred embodiment, the catalyst has the formula Mo_{1.0}V_{0.33}Ta_{0.12}Te_{0.28}O_{z}.

The following examples are included to demonstrate preferred embodiments of the invention. It should be appreciated by those of skill in the art that the techniques disclosed in the following examples represent techniques discovered by the inventors to function well in the practice of the invention, and thus can be considered to constitute preferred modes for its practice. However, those of skill in the art should appreciate, in light of the present disclosure, that many changes can be made in the specific embodiments which are disclosed and still obtain a like or similar result without departing from the scope of the invention.

### EXAMPLE

### Example 1

A catalyst having the formula Mo₁V_{0.33}Ta_{0.12}Te_{0.28}O_{z}, is prepared as follows: 25.0 g of ammonium heptamolybdate tetrahydrate (Aldrich Chemical Company), 5.47 g of ammonium metavanadate (Aldrich Chemical Company) and 9.10 g of telluric acid (Aldrich Chemical Company) are dissolved in 400 mL of water by heating to 80° C. After cooling to room temperature, 28.0 mL of an aqueous solution of tantalum oxalate (0.5 M Ta, 1.5 M oxalate) is added. The water is removed via a rotary evaporator with a warm water bath at 50° C. to obtain the catalyst precursor solid. The solid is dried at 120 C prior to calcination.

The catalyst precursor solid is calcined under a nitrogen atmosphere in a covered crucible pre-purged with nitrogen 600° C. for 2 hours. The oven is ramped at 10 deg C/min to % 0 C and held for 2 hours, and then reampned to 600 C at 10 C/min, and held at 600 C for 2 hours. The catalyst thus obtained is ground to a fine powder and pressed in a mold and then broken and sieved to 600-710 micron particles.

About 3 mL of the catalyst was mixed with about 7 mL of quartz particles and loaded into the bottom half of a stainless steel tube reactor with an internal diameter of 7.7 mm. Quartz is layered onto the top of the catalyst bed to both fill the reactor and to preheat the gaseous feeds prior to entering the catalyst bed. The reactor is heated and cooled by use of thermostated oil circulating in an external jacket. Water is vaporized in an evaporator and mixed with the desired volumes of ethane, oxygen, and nitrogen gases before being supplied to the reactor through mass flow controllers. The reaction pressure is maintained at the desired value by a back pressure regulator located on the reactor vent gas. The temperature in the catalyst bed is measured by a moveable thermocouple inserted in a thermowell in the center of the catalyst bed. The temperature is increased in the oil jacket until the desired oxygen conversion is achieved. The reaction feed gas and the product gas are analyzed on-line by gas chromatography.

The contact time is defined as:
t(sec)=bulk volume of the catalyst (mL)/a volume flow rate of the feed gas through the reactor at reaction conditions (mL/s).
GHSV=the gas hourly space velocity, is the reciprocal of the contact time, t, corrected to STP (0° C., 1 atm).

The ethane concentration in the feed was varied from 37 to 67 mol %, the oxygen concentration in the feed was varied from 7.6 to 15.3 mol %, and the water was varied from 4 to 9 mol %, with the balance being made up with nitrogen, as shown in Table 1. A very high selectivity to ethylene of 74 to 80% is achieved over a range of contact times, as shown in Table 2. Additionally, the selectivity to CO₂ and CO is very low, the sum never more than 8% over the range of conditions tested. Productivity as measured by the STY to ethylene is likewise very high with values as high as 460 kg ethylene per m³ per hour.

TABLE 1
Reaction Conditions

| | Reaction Conditions | | | | | | | | | |
|---|---|---|---|---|---|---|---|---|---|---|
| | Ethane | Ethylene | Oxygen | Nitrogen | Water | P | T | GHSV | | |
| Sample | (%) | (%) | (%) | (%) | (%) | (psig) | (sec) | (hr⁻¹) | T, Center | T, Shell |
| 1 | 39 | 0 | 8.1 | 43 | 5 | 220 | 10.2 | 2561 | 328 | na |
| 2 | 38 | 0 | 7.5 | 40 | 11 | 220 | 9.5 | 2732 | 318 | na |
| 3 | 37 | 0 | 8.4 | 49 | 9 | 216 | 9.7 | 2743 | 309 | 308 |
| 4 | 39 | 0 | 8.6 | 50 | 7 | 218 | 9.7 | 2746 | 309 | 308 |
| 5 | 38 | 0 | 8.7 | 53 | 3 | 217 | 9.6 | 2743 | 314 | 315 |
| 6 | 46 | 0 | 14.9 | 54 | 7 | 216 | 9.4 | 2738 | 323 | 320 |
| 7 | 38 | 0 | 15.3 | 41 | 5 | 215 | 9.3 | 2732 | 332 | 327 |
| 8 | 38 | 0 | 12.6 | 44 | 5 | 215 | 9.5 | 2742 | 320 | 319 |
| 9 | 40 | 0 | 14.4 | 41 | 4 | 215 | 14.4 | 1808 | 318 | 315 |
| 10 | 54 | 0 | 7.6 | 33 | 5 | 217 | 9.8 | 2740 | 305 | 303 |
| 11 | 66 | 0 | 7.8 | 19 | 5 | 217 | 10.0 | 2739 | 295 | 303 |
| 12 | 66 | 0 | 12.0 | 14 | 5 | 216 | 9.6 | 2737 | 315 | 312 |
| 13 | 65 | 0 | 15.1 | 11 | 5 | 215 | 9.4 | 2737 | 322 | 317 |
| 14 | 67 | 0 | 7.7 | 17 | 5 | 216 | 15.1 | 1814 | 290 | 291 |
| 15 | 67 | 0 | 12 | 13.6 | 4 | 216 | 14.8 | 1814 | 303 | 301 |
| 16 | 67 | 15 | 15 | 10.9 | 4 | 215 | 14.6 | 1813 | 310 | 307 |
| 17 | 66 | 14 | 14 | 15.5 | 0 | 216 | 14.4 | 1826 | 312 | na |

TABLE 2
Catalyst Performance

| | Ethane | | | | | |
|---|---|---|---|---|---|---|
| | Conv. | O2 Conv | Ethylene | CO₂ Sel | CO Sel | STY, |
| Sample | (%) | (%). | Sel (%) | (%) | (%) | Ethylene |
| 1 | 24 | 91 | 79 | 1 | 3 | 258 |
| 2 | 23 | 95 | 75 | 1 | 3 | 241 |
| 3 | 24 | 93 | 77 | 1 | 3 | 252 |
| 4 | 24 | 93 | 76 | 1 | 3 | 247 |
| 5 | 25 | 94 | 80 | 1 | 3 | 276 |
| 6 | 32 | 86 | 79 | 2 | 3 | 344 |
| 7 | 42 | 94 | 76 | 3 | 5 | 436 |
| 8 | 32 | 93 | 77 | 2 | 4 | 354 |
| 9 | 39 | 96 | 74 | 2 | 5 | 261 |
| 10 | 16 | 88 | 77 | 1 | 2 | 236 |
| 11 | 14 | 95 | 78 | 1 | 2 | 265 |
| 12 | 21 | 97 | 76 | 1 | 3 | 382 |
| 13 | 26 | 97 | 75 | 2 | 4 | 460 |
| 14 | 13 | 92 | 77 | 1 | 2 | 160 |
| 15 | 19 | 98 | 74 | 1 | 3 | 230 |
| 16 | 24 | 98 | 73 | 2 | 4 | 274 |
| 17 | 24 | 97 | 77 | 2 | 4 | 298 |

These results are a significant improvement compared to prior art. For example, the catalyst Mo_{2.5}V₁Nb_{0.32}Te_{1.69E-05} described in Example 10 of U.S. Pat. No. 6,013,957 produced only a 28.4% selectivity to ethylene, and while the selectivities to CO2 and CO were not reported, if it is assumed that the products not reported are indeed COx, then this inefficiency could be as high as 34.4%. Likewise, Example B of WO 2004/108277 reported only a 5% selectivity to ethylene for catalyst Mo₁V_{0.529}Nb_{0.124}Ti_{0.331}, with 35% selectivity to COₓ, So the present catalyst offers high selectivity to ethylene with much lower loss to the deep oxidation products, COₓ.

What is claimed is:

1. A process for preparing ethylene from a gaseous feed comprising ethane and oxygen, said process comprising contacting the gaseous feed with a catalyst in a reactor to produce an effluent comprising ethylene, the catalyst having the formula
MoₐVᵥTaₓTe_{y}O_{z}
wherein, a is 1.0, v is about 0.01 to about 1.0, x is about 0.01 to about 1.0, and y is about 0.01 to about 1.0, and z is the number of oxygen atoms necessary to render the catalyst electronically neutral.

2. The process of claim 1, wherein the gaseous feed further comprises ethylene.

3. The process of claim 1, wherein a is 1.0, v is about 0.1 to about 0.5, x is about 0.05 to about 0.2, and y is about 0.1 to about 0.5.

4. The process of claim 1, wherein A is Mo.

5. The process of claim 1, wherein X is Ta.

6. The process of claim 1, wherein Y is Te.

7. The process of claim 1, wherein the catalyst has the formula Mo_{1.0}V_{0.3}Ta_{0.1}Te_{0.3}O_{z}.

8. The process of claim 1, wherein the reactor is a fixed bed reactor containing the catalyst.

9. The process of claim 1, wherein the reactor is a fluidized bed reactor containing the catalyst.

10. The process of claim 1, wherein the gaseous feed contacts the catalyst at a temperature of about 200° C. to about 500° C.

11. The process of claim 10, wherein the gaseous feed contacts the catalyst at a temperature of about 200° C. to about 400° C.

12. The process of claim 1, wherein the catalyst is supported on a support selected from the group consisting of porous silicon dioxide, ignited silicon dioxide, kieselguhr, silica gel, porous and nonporous aluminum oxide, titanium dioxide, zirconium dioxide, thorium dioxide, lanthanum oxide, magnesium oxide, calcium oxide, barium oxide, tin oxide, cerium dioxide, zinc oxide, boron oxide, boron nitride, boron carbide, boron phosphate, zirconium phosphate, aluminum silicate, silicon nitride, silicon carbide, and glass, carbon, carbon-fiber, activated carbon, metal-oxide or metal networks and corresponding monoliths.

13. The process of claim 1, wherein the catalyst is not supported on a support.

14. The process of claim 1, wherein the catalyst is encapsulated in a material.

15. The process of claim 14, wherein the material is selected from the group consisting of SiO₂, P₂O₅, MgO, Cr₂O₃, TiO₂, ZrO₂, and Al₂O₃.

16. The process of claim 1, further comprising the step of separating a feed precursor comprising ethane and propane to provide the ethane.

17. The process of claim 1, wherein the effluent comprises carbon monoxide, further comprising the step of selectively oxidizing said effluent to convert the carbon monoxide to carbon dioxide.

18. The process of claim 17, further comprising the step of removing the carbon dioxide from the effluent.

19. The process of any one of claim 1, 16, or 18, further comprising the step of distilling the effluent to remove unreacted ethane therefrom.

20. The process of claim 19, further comprising the step of recycling the unreacted ethane to the reactor.

21. The process of any one of claim 1, 16, or 18, wherein the effluent comprises acetic acid, the process further comprising the step of separating the acetic acid from the effluent.

22. The process of claim 21, wherein the effluent comprises water, propionic acid, or a mixture thereof, the process further comprising the step of separating said water and said propionic acid from the acetic acid.

23. The process of any one of claim 1, 16, 17, or 18, further comprising the step of reacting the ethylene with acetic acid to produce vinyl acetate.

24. The process of claim 23, wherein at least some of the acetic acid is produced in the reactor.

25. The process of claim 1, wherein the catalyst has a selectivity for ethylene of about 50% to about 80%.

26. The process of claim 25, wherein the selectivity for ethylene is about 70% to about 80%.

27. A process for oxidizing ethane to produce ethylene and acetic acid, comprising contacting a catalyst with a gaseous feed comprising ethane and oxygen at a temperature of about 200° C. to about 400° C., wherein the catalyst has the formula Mo_{1.0}V_{0.33}Ta_{0.12}Te_{0.28}O_{z}.
